(19) United States
(12) Patent Application Publication
(10) Pub. No.: US 2006/0193813 A1
(43) Pub. Date: Aug. 31, 2006

(54) Nanoemulsion containing a hydroxylated urea compound

(75) Inventor: Jean-Thierry Simonnet, Cachan (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/350,826

(22) Filed: Feb. 10, 2006

Related U.S. Application Data
(60) Provisional application No. 60/655,915, filed on Feb. 25, 2005.

(30) Foreign Application Priority Data
Feb. 11, 2005 (FR) 05 01413

(51) Int. Cl. A61K 8/46

(52) U.S. Cl. 424/70.21; 424/70.31; 977/926

(57) ABSTRACT

The invention relates to an oil-in-water nanoemulsion having an oily phase dispersed in an aqueous phase, the oil globules of which have a number-average size of less than 100 nm, containing: (i) at least one amphiphilic lipid comprising at least one nonionic amphiphilic lipid, and optionally at least one ionic amphiphilic lipid, the oily phase and the amphiphilic lipid being present at a content such that the oily phase/amphiphilic lipid weight ratio ranges from 3 to 10, and (ii) at least one hydroxylated urea derivative. Application in cosmetics, dermatology, and ophthalmology.

### REFERENCE TO PRIOR APPLICATIONS

This application claims priority to U.S. provisional application 60/655,915 filed Feb. 25, 2005, and to French patent application 0501413 filed Feb. 11, 2005, both incorporated herein by reference.

### FIELD OF THE INVENTION

The present invention relates to a nanoemulsion comprising at least one nonionic amphiphilic lipid, optionally at least one anionic amphiphilic lipid, and at least one urea compound, and to the use of this nanoemulsion in, e.g., topical application, in particular in the cosmetic and dermatological fields, and in the pharmaceutical and/or ophthalmological fields.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Oil-in-water (O/W) emulsions are well known in the cosmetics and dermatology fields, in particular for the preparation of cosmetic products such as milks, creams, tonics, serums or eaux de toilette.

Nanoemulsions are O/W emulsions characterized by a size of the oily globules of less than 100 nm, the oily globules being stabilized by a wreath of amphiphilic lipids, which can optionally form a liquid crystal phase of lamellar type, located at the oil/aqueous phase interface. The transparency of these emulsions comes from the small size of the oily globules, a small size obtained by virtue of the use of mechanical energy and in particular of a high-pressure homogenizer. Nanoemulsions are to be distinguished from microemulsions by their structure. This is because microemulsions are thermodynamically stable dispersions consisting of amphiphilic lipid micelles which are swollen by oil. Furthermore, microemulsions do not require high mechanical energy to be prepared; they form spontaneously by simply bringing the constituents into contact. The major drawbacks of microemulsions are related to their high proportion of surfactants, resulting in intolerance and leading to a sticky feel when applied to the skin. Moreover, their formulation range is generally very narrow and their temperature stability very limited.

Nanoemulsions comprise one or more amphiphilic lipid(s). The term “amphiphilic lipid” is intended to mean here any molecules having a bipolar structure, i.e. comprising at least one hydrophobic part and at least one hydrophilic part, and having the property of reducing the surface tension of water (γ<55 mN/m) and of reducing the interfacial tension between water and an oily phase. Synonyms for amphiphilic lipid are, for example: surfactants, surface-active agent, emulsifier.

Documents EP-A-728 460 and EP-A-780 114 describe nanoemulsions based on liquid nonionic amphiphilic lipids or on silicone surfactants. Nanoemulsions are also described in documents FR-A-2,787,026, FR-A-2,787,027, FR-A-2,787,325,

FR-A-2,787,326, FR-A-2,787,703, FR-A-2,787,728.

In order to promote the production of oil droplets having a size less than 100 nm, solvents such as ethanol or glycols, for instance propylene glycol, butylene glycol or dipropylene glycol, are used in a known manner. Now, these solvents have the disadvantage of conferring poor cosmetic properties on the nanoemulsion, essentially due to the sticky nature of the nanoemulsion, noticed in particular when applied to the skin. In addition, these nanoemulsions do not exhibit any satisfactory skin-moisturizing property.

### SUMMARY OF THE INVENTION

One aim of the present invention is therefore to have nanoemulsions that are stable, in particular for one month at 45° C., and that do not exhibit any sticky effect, especially when they are applied to keratin materials, and in particular to the skin. Another aim of the invention is to have a nanoemulsion that has a good skin-moisturizing property.

The inventors have discovered that such compositions are obtained by using a specific urea compound in the nanoemulsion. This urea compound makes it possible to prepare nanoemulsions that are stable, in particular for one month at 45° C. The nanoemulsions obtained do not exhibit any sticky effect, and make it possible to moisturize the skin.

### DETAILED DESCRIPTION OF THE PREFERRED EMBODIMENTS

One subject of the invention is an oil-in-water nanoemulsion comprising an oily phase dispersed in an aqueous phase, the oil globules of which have a number-average size of less than 100 nm, wherein it comprises:

(i) at least one amphiphilic lipid comprising at least one nonionic amphiphilic lipid, and optionally at least one ionic amphiphilic lipid, the oily phase and the amphiphilic lipid being present in a content such that the oily phase/amphiphilic lipid weight ratio is 3-10, and

(ii) at least one specific urea compound.

The nanoemulsions according to the invention generally have a transparent to bluish appearance. Their transparency measured by a transmittance coefficient at 600 nm preferably ranges from 10% to 90%, or else by a turbidity. The turbidity of the compositions of the invention preferably ranges from 60 to 400 NTU, and more preferably from 70 to 300 NTU, the turbidity being measured with a Hach portable turbidity meter—model 2100 P, at approximately 25° C.

The oil globules of the nanoemulsions of the invention preferably have a number-average size of less than 100 nm, more preferably ranging from 20 to 80 nm, and even more preferably from 40 to 60 nm. While not bound by theory, it is believed that the decrease in the size of the globules makes it possible to promote penetration of the active agents into the superficial layers of the skin (carrier effect).

The nanoemulsions in accordance with the invention are preferably prepared at temperatures ranging from 4 to 45° C. and are thus compatible with thermosensitive active agents.

The urea compound present in the composition according to the invention is at least one compound of formula (I) below: 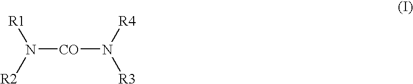
in which:
R₁, R₂, R₃ and R₄ each represent, independently of one another, a hydrogen atom, a C₁-C₄ alkyl group or a C₂-C₆ hydroxyalkyl group that may contain from 1 to 5 hydroxyl groups, where at least one of the radicals R₁ to R₄ represents a hydroxyalkyl group, and also the salts, solvates and isomers thereof.

For the compounds of formula (I):

preferably, R₁ denotes a C₂-C₆ hydroxyalkyl group, and R₂, R₃ and R₄ denote, independently of one another, a hydrogen atom or a C₁-C₄ alkyl group;

more preferably, R₁ denotes a C₂-C₆ hydroxyalkyl group containing from 1 to 5 hydroxyl groups, in particular 1 hydroxyl group, and R₂, R₃ and R₄ denote a hydrogen atom;

even more preferably, R₁ denotes a C₂-C₄ hydroxyalkyl group containing 1 hydroxyl group, and R₂, R₃ and R₄ denote a hydrogen atom.

Among the alkyl groups, mention may be made of methyl, ethyl, n-propyl, isopropyl, n-butyl, isobutyl and tert-butyl groups.

Among the hydroxyalkyl groups, those containing a single hydroxyl group, and in particular hydroxyethyl, hydroxypropyl, hydroxybutyl, hydroxypentyl and hydroxyhexyl groups, are preferred.

Among the salts, mention may be made of salts of inorganic acids, such as sulphuric acid, hydrochloric acid, hydrobromic acid, hydriodic acid, phosphoric acid or boric acid. Mention may also be made of salts of organic acids, which may contain one or more carboxylic, sulphonic or phosphonic acid groups. They may be linear, branched or cyclic aliphatic acids, or alternatively aromatic acids. These acids may also contain one or more hetero atoms chosen from O and N, for example in the form of hydroxyl groups. Mention may in particular be made of propionic acid, acetic acid, terephthalic acid, citric acid and tartaric acid.

The term “solvate” is intended to mean a stoichiometric mixture of said compound of formula (I) with one or more molecules of water or of organic solvent, such a mixture being derived from the synthesis of the compound of formula (I).

As preferred compounds of formula (I), mention may be made of N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxy-hexyl)urea; N-methyl-N-(1,3,4,5,6-pentahydroxy-2-hexyl)urea; N-methyl-N′-(1-hydroxy-2-methyl-2-propyl)-urea; N-(1-hydroxy-2-methyl-2-propyl)urea; N-(1,3-hydroxy-2-propyl)urea; N-(trishydroxymethylmethyl)-urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxy-propyl)urea; N,N-bis(2-hydroxyethyl)-N′-propylurea; N,N-bis(2-hydroxypropyl)-N′-(2-hydroxyethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof.

Preferably, the compound of formula (I) is N-(2-hydroxyethyl)urea.

The compounds of formula (I) are compounds that are known and in particular described in application DE-A-2703185. Among these, N-(2-hydroxy-ethyl)urea is also commercially available, in the form of a 50% mixture by weight in water, from the company National Starch under the trade name Hydrovance®.

The compound of formula (I) may be present in the nanoemulsion according to the invention in any amount including at a content ranging from 1% to 50% by weight, relative to the total weight of the composition, preferably ranging from 2% to 25% by weight, and preferentially ranging from 2% to 20% by weight.

The nanoemulsions according to the invention comprise at least one nonionic amphiphilic lipid.

The nonionic amphiphilic lipids of the invention are preferably chosen from:

1/silicone surfactants,

2/amphiphilic lipids that are liquid at a temperature of less than or equal to 45° C., chosen from esters of at least one polyol and of at least one fatty acid comprising at least one saturated or unsaturated, linear or branched, and in particular unsaturated or branched, C₈-C₂₂ alkyl chain, the polyol being chosen from the group formed by polyethylene glycol comprising from 1 to 60 ethylene oxide units, sorbitan, glycerol possibly comprising from 2 to 30 ethylene oxide units, and polyglycerols comprising from 2 to 15 glycerol units,

3/fatty acid esters of a sugar and fatty alcohol ethers of a sugar,

4/surfactants that are solid at a temperature equal to 45° C., chosen from glycerol fatty esters, sorbitan fatty esters and oxyethylenated sorbitan fatty esters, ethoxylated fatty ethers and ethoxylated fatty esters,

5/block copolymers of ethylene oxide (A) and of propylene oxide (B),

and mixtures of these surfactants.

1/The silicone surfactants that can be used according to the invention are silicone compounds comprising at least one oxyethylene —OCH₂CH₂— chain and/or oxypropylene —OCH₂CH₂C₂— chain. As silicone surfactants that may be used according to the present invention, mention may be made of those described in documents U.S. Pat. No. 5,364,633 and U.S. Pat. No. 5,411,744.

Preferably, the silicone surfactant used according to the present invention is a compound of formula (II): 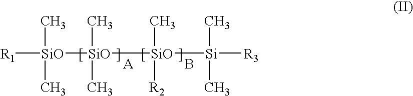
in which:
R₁, R₂, R₃, independently of one another, represent a C₁-C₆ alkyl radical or a (CH₂)ₓ—(OCH₂CH₂)_{y}—(OCH₂CH₂CH₂)_{z}—OR₄ radical, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; on the condition that A and B are not equal to zero at the same time;
x is an integer ranging from 1 to 6;
y is an integer ranging from 1 to 30;
z is an integer ranging from 0 to 5.

According to a preferred embodiment of the invention, in the compound of formula (X), the alkyl radical is a methyl radical, x is an integer ranging from 2 to 6 and y is an integer ranging from 4 to 30.

By way of example of silicone surfactants of formula (II), mention may be made of the compounds of formula (III): 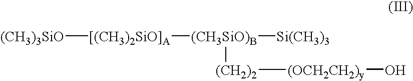
in which A is an integer ranging from 20 to 105, B is an integer ranging from 2 to 10 and y is an integer ranging from 10 to 20.

Mention may also be made, by way of example of silicone surfactants of formula (II), of the compounds of formula (IV):
H—(OCH₂CH₂)_{y}—(CH₂)₃—[(CH₃)₂SiO]_{A}—(CH₂)₃—(OCH₂CH₂)_{y}—OH (IV)
in which A′ and y are integers ranging from 10 to 2.0.

Use may in particular be made, as silicone surfactants, of those sold by Dow Corning under the names DC 5329, DC 7439-146, DE 2-5695 and Q4-3667. The compounds DC 5329, DC 7439-146 and DC 2-5695 are compounds of formula (XI) where, respectively, A is 22, B is 2 and y is 12; A is 103, B is 10 and y is 12; and A is 27, B is 3 and y is 12.

The compound Q4-3667 is a compound of formula (IV) where A is 15 and y is 13.

2/The amphiphilic lipids that are liquid at a temperature of less than or equal to 45° C. can be chosen in particular from:

polyethylene glycol isostearate with a molecular weight of 400 (CTFA name: PEG-8 isostearate), sold under the name Prisorine 3644 by the company Unichemia;

diglyceryl isostearate, sold by the company Solvay;

polyglyceryl laurate comprising 2 glycerol units (polyglyceryl-2 laurate), sold under the name diglycerin monolaurate by the company Solvay;

sorbitan oleate, sold under the name Span 80 by the company ICI;

sorbitan isostearate, sold under the name Nikkol SI 10R by the company Nikko;

α-butylglucoside cocoate or α-butylglucoside caprate, sold by Ulice.

3/The fatty acid esters of a sugar that can be used as nonionic amphiphilic lipids in the nanoemulsion according to the invention are preferably solid at a temperature of less than or equal to 45° C. and can be chosen in particular from the group comprising esters or mixtures of esters of a C₈-C₂₂ fatty acid and of sucrose, of maltose, of glucose or of fructose, and esters or mixtures of esters of a C₁₄-C₂₂ fatty acid and of methylglucose.

The C₈-C₂₂ or C₁₄-C₂₂ fatty acids forming the fatty unit of the esters that can be used in the nanoemulsion of the invention comprise a saturated or unsaturated linear alkyl chain having, respectively, from 8 to 22 or from 14 to 22 carbon atoms. The fatty unit of the esters can be chosen in particular from stearates, behenates, arachidonates, palmitates, myristates, laurates, caprates and mixtures thereof. Stearates are preferably used.

By way of example of esters or of mixtures of esters of a fatty acid and of sucrose, of maltose, of glucose or of fructose, mention may be made of sucrose monostearte, sucrose distearate, sucrose tristearate and mixtures thereof, such as the products sold by the company Croda under the name Crodesta F50, F70, F110 and F160 having, respectively, an HLB (Hydrophilic Lipophilic Balance) of 5, 7, 11 and 16; and, by way of example of esters or of mixtures of esters of a fatty acid and of methylglucose, mention may be made of the disearate of methylglucose and of polyglycerol-3, sold by the company Goldschmidt under the name Tego-care 450. Mention may also be made of glucose monoesters or maltose monoesters, such as methyl O-hexadecanoyl-6-D-glucoside and O-hexadecanoyl-6-D-maltoside.

The fatty alcohol ethers of a sugar that can be used as nonionic amphiphilic lipids in the nanoemulsion according to the invention are solid at a temperature of less than or equal to 45° C. and can be chosen in particular form the group comprising ethers or mixtures of ethers of a C₈-C₂₂ fatty alcohol and of glucose, of maltose, of sucrose or of fructose, and ethers or mixtures of ethers of a C₁₄-C₂₂ fatty alcohol and of methylglucose. They are in particular alkylpolyglucosides.

The C₈-C₂₂ or C₁₄-C₂₂ fatty alcohols forming the fatty unit of the ethers that can be used in the nanoemulsion of the invention comprise a saturated or unsaturated linear alkyl chain having, respectively, from 8 to 22 or from 14 to 22 carbon atoms. The fatty unit of the ethers can be chosen in particular from decyl, cetyl, behenyl, arachidyl, stearyl, palmityl, myristyl, lauryl, capryl and hexadecanoyl units, and mixtures thereof such as cetearyl.

By way of example of fatty alcohol ethers of a sugar, mention may be made of alkylpolyglucosides, such as decylglucoside and laurylglucoside sold, for example, by the company Henkel under the respective names Plantaren 2000 and Plantaren 1200, cetostearylglucoside, optionally as a mixture with cetostearyl alcohol, sold, for example, under the name Montanov 68 by the company Seppic, under the name Tego-care CG90 by the company Goldschmidt and under the name Emulgade KE3302 by the company Henkel, and also arachidylglucoside, for example in the form of the mixture of arachidyl and behenyl alcohols and of arachidylglucoside sold under the name Montanov 202 by the company Seppic.

Use is more particularly made, as nonionic amphiphilic lipid of this type, of sucrose monostearate, sucrose distearate, sucrose tristearate and mixtures thereof, the distearate of methylglucose and of polyglycerol-3, and alkylpolyglucosides.

4/The glycerol fatty esters that can be used as nonionic amphiphilic lipids in the nanoemulsion according to the invention, that are solid at a temperature equal to 45° C., can be chosen in particular from the group comprising the esters formed from at least one acid comprising a saturated linear alkyl chain having from 16 to 22 carbon atoms, and from 1 to 10 glycerol units. Use may be made of one or more of these glycerol fatty esters in the nanoemulsion of the invention.

These esters may be chosen in particular from stearates, behenates, arachidates, palmitates and mixtures thereof. Stearates and palmitates are preferably used.

By way of example of a surfactant that can be used in the nanoemulsion of the invention, mention may be made of decaglycerol monostearate, distearate, tristearate and pentastearate (10 glycerol units) (CTFA names: polyglyceryl-10 stearate, polyglyceryl-10 distearate, polyglyceryl-10 tristearate, polyglyceryl-10 pentastearate), such as the products sold under the respective names Nikkol Decaglyn 1-S, 2-S, 3-S and 5-S by the company Nikko, and diglyceryl monostearate (CTFA name: polyglyceryl-2 stearate) such as the product sold by the company Nikko under the name Nikkol DGMS.

The sorbitan fatty esters that can be used as nonionic amphiphilic lipids in the nanoemulsion according to the invention, that are solid at a temperature of less than or equal to 45° C., are chosen in particular from the group comprising esters of a C₁₆-C₂₂ fatty acid and of sorbitan and oxyethylenated esters of a C₁₆-C₂₂ fatty acid and of sorbitan. They are formed from at least one fatty acid comprising at least one saturated linear alkyl chain, having, respectively, from 16 to 22 carbon atoms, and from sorbitol or from ethoxylated sorbitol. The oxyethylenated esters generally comprise from 1 to 100 ethylene oxide units, and preferably from 2 to 40 ethylene oxide (EO) units.

These esters can be chosen in particular from stearates, behenates, arachidates, palmitates and mixtures thereof. Stearates and palmitates are preferably used.

By way of example of sorbitan fatty ester and of an oxyethylenated sorbitan fatty ester, that can be used in a nanoemulsion of the invention, mention may be made of sorbitan monostearate (CTFA name: sorbitan stearate) sold by the company ICI under the name Span 60, sorbitan monopalmitate (CTFA name: sorbitan palmitate) sold by the company ICI under the name Span 40, or sorbitan 20 EO tristearate (CTFA name: polysorbate 65) sold by the company ICI under the name Tween 65.

The ethoxylated fatty ethers that are solid at a temperature of less than or equal to 45° C., that can be used as nonionic amphiphilic lipids in the nanoemulsion according to the invention, are preferably ethers made up of 1 to 100 ethylene oxide units and of at least one fatty alcohol chain having from 16 to 22 carbon atoms. The fatty chain of the ethers can be chosen in particular from behenyl, arachidyl, stearyl and cetyl units, and mixtures thereof, such as cetearyl. By way of example of ethoxylated fatty ethers, mention may be made of ethers of behenyl alcohol comprising 5, 10, 20 and 30 ethylene oxide units (CTFA names: beheneth-5, beheneth-10, beheneth-20 and beheneth-30), such as the products sold under the names Nikkol BB5, BB10, BB20 and BB30 by the company Nikko, and the ether of stearyl alcohol comprising 2 ethylene oxide units (CTFA name: steareth-2), such as the product sold under the name Brij 72 by the company ICI.

The ethoxylated fatty esters that are solid at a temperature of less than or equal to 45° C., that can be used as nonionic amphiphilic lipids in the nanoemulsion according to the invention, are esters made up of 1 to 100 ethylene oxide units and of at least one fatty acid chain comprising from 16 to 22 carbon atoms. The fatty chain of the esters can be chosen in particular from stearate, behenate, arachidate and palmitate units, and mixtures thereof. By way of example of ethoxylated fatty esters, mention may be made of the ester of stearic acid comprising 40 ethylene oxide units, such as the product sold under the name Myrj 52 (CTFA name: PEG-40 stearate) by the company ICI, and the ester of behenic acid comprising 8 ethylene oxide units (CTFA name: PEG-8 behenate), such as the product sold under the name Compritol HD5 ATO by the company Gattefosse.

5/The block copolymers of ethylene oxide and of propylene oxide that can be used as nonionic amphiphilic lipids in the nanoemulsion according to the invention can be chosen in particular from the block copolymers of formula (V):
HO(C₂H₄O)x(C₃H₆O)y(C₂H₄O)zH (V)
in which x, y and z are integers such that x+z ranges from 2 to 100 and y ranges from 14 to 60, and mixtures thereof, and more particularly from the block copolymers of formula (V) having an HLB ranging from 2 to 16.

These block copolymers can be chosen in particular from poloxamers and in particular from Poloxamer 231, such as the product sold by the company ICI under the name Pluronic L81 of formula (V) with x=z=6, y=39 (HLB 2); Poloxamer 282, such as the product sold by the company ICI under the name Pluronic L92 of formula (V) with x=z=10, y=47 (HLB 6); and Poloxamer 124, such as the product sold by the company ICI under the name Pluronic L44 of formula (V) with x=z=11, y=21 (HLB 16).

As nonionic amphiphilic lipids, mention may also be made of the mixtures of nonionic surfactants described in document EP-A-705593, incorporated herein for reference.

Among the nonionic amphiphilic lipids, use may in particular be made of:

PEG-400 isostearate or PEG-8 isostearate (comprising 8 mol of ethylene oxide),

diglyceryl isostearate,

polyglyceryl monolaurate comprising 2 glycerol units and polyglyceryl stearate comprising 10 glycerol units,

sorbitan oleate,

sorbitan isostearate,

and mixtures thereof.

The nonionic amphiphilic lipids may be present in the nanoemulsion according to the invention at a content ranging from 0.2% to 12% by weight, relative to the total weight of the composition, and preferably ranging from 0.2% to 8% by weight, and preferentially ranging from 0.2% to 6% by weight.

According to a particular embodiment of the invention, the nanoemulsion of the invention may also contain one or more ionic amphiphilic lipids, in particular one or more anionic or cationic lipids, that are different from the nonionic amphiphilic lipids described above. The addition thereof may further improve the stability of the dispersion.

Thus, the anionic amphiphilic lipids that can be used in the nanoemulsions of the invention are preferably chosen from:

alkali metal salts of dicetyl phosphate and of dimyristyl phosphate;

alkali metal salts of cholesterol sulphate;

alkali metal salts of cholesterol phosphate;

lipoamino acids and their salts, such as mono and disodium acylglutamates, for instance the disodium salt of N-stearoyl-L-glutamic acid sold under the name Acylglutamate HS21 by the company Ajinomoto;

sodium salts of phosphatidic acid;

phospholipids;

alkylsulphonic derivatives, in particular of formula (VI): 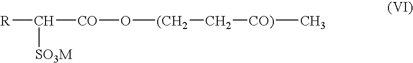
in which R represents C₁₆-C₂₂ alkyl radicals, in particular the C₁₆H₃₃ and C₁₈H₃₇ radicals taken as a mixture or separately, and M is an alkali metal or alkaline earth metal, such as sodium; and mixtures thereof.

The cationic amphiphilic lipids that can be used in the nanoemulsions of the invention are preferably chosen from the group formed by quaternary ammonium salts, and fatty amines and their salts.

The quaternary ammonium salts are, for example:

those that have the general formula (VII) below: 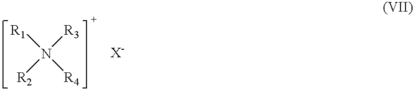
in which the radicals R₁ to R₄, which may be identical or different, represent a linear or branched aliphatic radical comprising from 1 to 30 carbon atoms, or an aromatic radical such as aryl or alkylaryl. The aliphatic radicals can comprise hetero atoms, such as, in particular, oxygen, nitrogen or sulphur, or halogens. The aliphatic radicals are, for example, chosen from alkyl, alkoxy, poloxy(C₂-C₆)alkylene, alkylamide, (C₁₂-C₂₂)alkylamido(C₂-C₆)alkyl, (C₁₂-C₂₂)alkyl acetate or hydroxyalkyl radicals comprising approximately from 1 to 30 carbon atoms; X is an anion chosen from the group of halides, phosphates, acetates, lactates, (C₂-C₆)alkyl sulphates and alkyl- or alkylarylsulphonates.

Among the quaternary ammonium salts of formula (VIII), preference is given, firstly, to tetraalkylammonium chlorides, such as, for example, dialkyldimethylammonium or alkyltrimethylammonium chlorides in which the alkyl radical comprises approximately from 12 to 22 carbon atoms, in particular behenyltrimethylammonium, distearyldimethylammonium, cetyltrimethylammonium or benzyldimethylstearylammonium chloride, or alternatively, secondly, stearamidopropyl-dimethyl(myristyl acetate) ammonium chloride, sold under the name “Ceraphyl 70” by the company Van Dyk. Behenyltrimethylammonium chloride is the quaternary ammonium salt most particularly preferred;

imidazolinium quaternary ammonium salts, such as, for example, that having formula (VIII) below: 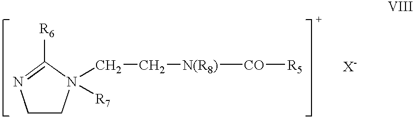
in which R₅ represents an alkenyl or alkyl radical comprising from 8 to 30 carbon atoms, for example derived from tallow fatty acids, R₆ represents a hydrogen atom, a C₁-C₄ alkyl radical or an alkenyl or alkyl radical comprising from 8 to 30 carbon atoms, R₇ represents a C₁-C₄ alkyl radical, R₈ represents a hydrogen atom or a C₁-C₄ alkyl radical, and X is an anion chosen from the group of halides, phosphates, acetates, lactates, alkyl sulphates, and alkyl- or alkylarylsulphonates. Preferably, R₅ and R₆ denote a mixture of alkenyl or alkyl radicals comprising from 12 to 21 carbon atoms, for example derived from tallow fatty acids, R₇ denotes methyl, and R₈ denotes hydrogen. Such a product is, for example, sold under the name “Rewoquat W75” by the company Rewo;

quaternary diammonium salts of formula (IX): 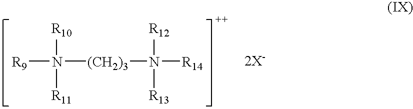
in which R₉ denotes an aliphatic radical comprising approximately from 16 to 30 carbon atoms, R₁₀, R₁₁, R₁₂, R₁₃ and R₁₄, which may be identical or different, are chosen from hydrogen or an alkyl radical comprising from 1 to 4 carbon atoms, and X is an anion chosen from the group of halides, acetates, phosphates, nitrates and methyl sulphates. Such quaternary diammonium salts comprise in particular propanetallowdiammonium dichloride;

quaternary ammonium salts containing at least one ester function.

The quaternary ammonium salts containing at least one ester function that can be used according to the invention are, for example, those of formula (X) below: 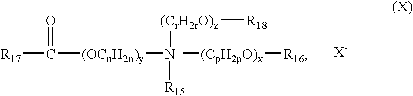
in which:

R₁₅ is chosen from C₁-C₆ alkyl radicals and C₁-C₆ hydroxyalkyl or dihydroxyalkyl radicals;

R₁₆ is chosen from:
the 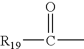
   radical
linear or branched, saturated or unsaturated C₁-C₂₂ hydrocarbon-based R₂₀ radicals,
a hydrogen atom,

R₁₈ is chosen from:
the 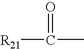
   radical
linear or branched, saturated or unsaturated C₁-C₆ hydrocarbon-based R₂₂ radicals,
a hydrogen atom,

R₁₇, R₁₉ and R₂₁, which may be identical or different, are chosen from linear or branched, saturated or unsaturated C₇-C₂₁ hydrocarbon-based radicals;

n, p and r, which may be identical or different, are integers of from 2 to 6;

y is an integer of from 1 to 10;

x and z, which may be identical or different, are integers of from 0 to 10;

X⁻ is a simple or complex, organic or inorganic anion; with the proviso that the sum x+y+z is from 1 to 15, that, when x is 0, then R₁₆ denotes R₂₀, and that, when z is 0, then R₁₈ denotes R₂₂.

The R₁₅ alkyl radicals may be linear or branched, and more particularly linear.

Preferably, R₁₅ denotes a methyl, ethyl, hydroxyethyl or dihydroxypropyl radical, and more particularly a methyl or ethyl radical.

Advantageously, the sum x+y+z is from 1 to 10.

When R₁₆ is a hydrocarbon-based R₂₀ radical, it may be long and have from 12 to 22 carbon atoms, or short and have from 1 to 3 carbon atoms.

When R₁₈ is a hydrocarbon-based R₂₂ radical, it preferably has 1 to 3 carbon atoms.

Advantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z which may be identical or different, are 0 or 1.

Advantageously, y is equal to 1.

Preferably, n, p and r, which may be identical or different, are 2 or 3, and even more particularly are equal to 2.

In formula (X), the anion X⁻ is preferably a halide (chloride, bromide or iodide) or an alkyl sulphate, more particularly methyl sulphate. Use may, however, be made of methanesulphonate, phosphate, nitrate, tosylate, an anion derived from an organic acid, such as acetate or lactate, or any other anion compatible with ammonium containing an ester function. The anion X⁻ is even more particularly chloride or methyl sulphate.

Use is more particularly made of ammonium salts of formula (X) in which:

R₁₅ denotes a methyl or ethyl radical;

x and y are equal to 1;

z is equal to 0 or 1;

n, p and r are equal to 2;

R₁₆ is chosen from:
the 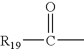
   radical;
methyl, ethyl or C₁₄-C₂₂ hydrocarbon-based radicals;
a hydrogen atom;

R₁₈ is chosen from:
the 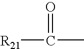
   radical,
a hydrogen atom;
   R₁₇, R₁₉ and R₂₁, which may be identical or different, are chosen from linear or branched, saturated or unsaturated C₁₃-C₁₇ hydrocarbon-based radicals; and preferably from linear or branched, saturated or unsaturated C₁₃-C₁₇ alkyl and alkenyl radicals.

Advantageously, the hydrocarbon-based radicals are linear.

As compounds of formula (X), mention may, for example, be made of diacyloxyethyldimethylammonium, diacyloxyethyl(hydroxyethyl)methylammonium, monoacyloxyethyl(dihydroxyethyl)methylammonium, triacyloxyethylmethylammonium or monoacyloxyethyl-(hydroxyethyl)dimethylammonium salts (chloride or methyl sulphate in particular), and mixtures thereof. The acyl radicals preferably have 14 to 18 carbon atoms and originate more particularly from a plant oil such as palm oil or sunflower oil. When the compound contains several acyl radicals, the latter may be identical or different. These products are obtained, for example, by direct esterification of triethanol-amine, of triisopropanolamine, of alkyldiethanolamine or of alkyldiisopropanolamine, which are optionally oxyalkylenated, with fatty acids or with mixtures of fatty acids of plant or animal origin, or by transesterification of their methyl esters. This esterification is followed by quaternization using an alkylating agent such as an alkyl (preferably methyl or ethyl) halide, a dialkyl (preferably methyl or ethyl) sulphate, methyl methanesulphonate, methyl para-toluenesulphonate, glycol chlorohydrin or glycerol chlorohydrin.

Such compounds are sold, for example, under the names Dehyquart by the company Henkel, Stepanquat by the company Stepan, Noxamium by the company Ceca, and Rewoquat WE 18 by the company Rewo-Witco.

When it contains ammonium salts, the composition according to the invention preferably contains a mixture of quaternary ammonium mono-, di- and triester salts with a majority by weight of diester salts.

As a mixture of ammonium salts, use may be made, for example, of the mixture containing 15 to 30% by weight of acyloxyethyl(dihydroxyethyl)methylammonium methyl sulphate, 45 to 60% of diacyloxyethyl-(hydroxyethyl)methylammonium methyl sulphate and 15 to 30% of triacyloxyethylmethylammonium methyl sulphate, the acyl radicals having from 14 to 18 carbon atoms and originating from palm oil which is optionally partially hydrogenated.

Use may also be made of the ammonium salts containing at least one ester function described in U.S. Pat. No. 4,874,554 and U.S. Pat. No. 4,137,180.

When the nanoemulsion contains one or more ionic amphiphilic lipids, they are present in the nanoemulsions of the invention, preferably, in concentrations ranging from 0.01 to 6% by weight relative to the total weight of the nanoemulsion, and more particularly from 0.2 to 4% by weight.

Depending on whether it is more hydrophilic or more lipophilic in nature, the nonionic or ionic amphiphilic lipid may be introduced into the aqueous phase or into the oily phase of the nanoemulsion. The total content of nonionic and ionic amphiphilic lipids can preferably range from 0.25 to 15% by weight, and preferably from 1 to 10% by weight, relative to the total weight of the nanoemulsion.

The oily phase of the nanoemulsion according to the invention comprises at least one oil. The oils that can be used in the nanoemulsions of the invention are preferably chosen from the group formed by:

animal or plant oils formed by fatty acid esters of polyols, in particular liquid triglycerides, for example sunflower oil, corn oil, soybean oil, avocado oil, jojoba oil, marrow oil, grapeseed oil, sesame oil, hazelnut oil, fish oils, glyceryl tricaprocaprylate, or plant or animal oils of formula R₉COOR₁₀ in which R₉ represents a higher fatty acid residue containing from 7 to 29 carbon atoms and R₁₀ represents a linear or branched hydrocarbon-based chain containing from 3 to 30 carbon atoms, in particular alkyl or alkenyl, for example purcellin oil or liquid jojoba wax;

natural or synthetic essential oils such as, for example, eucalyptus oil, lavandin oil, lavender oil, vetivier oil, litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;

synthetic oils such as parleam oil, polyolefins and liquid carboxylic acid esters;

mineral oils, such as hexadecane, isohexadecane and liquid paraffin;

halogenated oils, in particular fluorocarbons, such as fluoroamines, for example perfluorotributylamine, fluorinated hydrocarbons, for example perfluoro-decahydronaphthalene, fluoroesters and fluoroethers;

volatile and non-volatile silicone oils.

The polyolefins that can be used as synthetic oils are in particular poly-α-olefins, and more particularly those of hydrogenated or nonhydrogenated polybutene type, and preferably hydrogenated or nonhydrogenated polyisobutene type.

The liquid carboxylic acid esters that can be used as synthetic oils may be esters of mono-, di-, tri- or tetracarboxylic acids. The total carbon number of the esters is generally greater than or equal to 10, and preferably less than 100, and more particularly less than 80. They are in particular monoesters of saturated or unsaturated, linear or branched C₁-C₂₆ aliphatic acids and of saturated or unsaturated, linear or branched C₁-C₂₆ aliphatic alcohols, the total carbon number of the esters generally being greater than or equal to 10. Use may also be made of esters of C₄-C₂₂ di- or tricarboxylic acids and of C₁-C₂₂ alcohols, and esters of mono-, di- or tricarboxylic acids and of C₂-C₂₆ di-, tri-, tetra- or pentahydroxylated alcohols.

Among the abovementioned esters, use is preferably made of alkyl palmitates, such as ethyl palmitate, isopropyl palmitate, 2-ethylhexyl palmitate, 2-octyldecyl palmitate; alkyl myristates, such as isopropyl myristate, butyl myristate, cetyl myristate, 2-octyldodecyl myristate; alkyl stearates, such as hexyl stearate, butyl stearate or isobutyl stearate; alkyl malates, such as dioctyl malate; alkyl laurates, such as hexyl laurate and 2-hexyldecyl laurate; isononyl isononanoate; or cetyl octanoate.

Advantageously, the nanoemulsion according to the invention contains at least one oil of molecular weight greater than or equal to 400, in particular ranging from 400 to 10 000, better still ranging from 400 to 5000, or alternatively ranging from 400 to 5000. The oils of molecular weight greater than or equal to 400 can be chosen from oils of animal or plant origin, mineral oils, synthetic oils and silicone oils, and mixtures thereof. As oils of this type, mention may, for example, be made of isocetyl palmitate, isocetyl stearate, avocado oil and jojoba oil.

The nanoemulsions in accordance with the invention comprise an amount of oily phase (oil and other fatty substances apart from the amphiphilic lipid) preferably ranging from 2 to 40% by weight relative to the total weight of the nanoemulsion, and more particularly from 4 to 30% by weight, and preferably from 4 to 20% by weight.

According to a particular embodiment of the invention, the nanoemulsions in accordance with the invention comprise an oily phase comprising a proportion of oil(s) having a molecular weight of greater than or equal to 400 that preferably represents at least 40% by weight of the oily phase.

The oily phase and the amphiphilic lipids (nonionic and ionic amphiphilic lipids) are preferably present in the nanoemulsion according to the invention according to a weight ratio of the amount of oily phase to the amount of amphiphilic lipid ranging from 3 to 10, and preferably ranging from 3 to 6. The expression “amount of oily phase” is here intended to mean the total amount of the constituents of this oily phase without including the amount of amphiphilic lipid.

The nanoemulsions in accordance with the present invention may additionally contain, in addition to the urea derivatives of formula (I) described above, solvents, in particular for improving, if necessary, the transparency of the composition.

The solvents are preferably chosen from the group formed by:

lower C₁-C₈ alcohols, such as ethanol;

glycols, such as glycerol, propylene glycol, 1,3-butylene glycol, dipropylene glycol or polyethylene glycols comprising from 4 to 16 ethylene oxide units, and preferably from 8 to 12;

sugars, such as glucose, fructose, maltose, lactose or sucrose.

These solvents may be used as a mixture. When they are present in the nanoemulsion of the invention, they can be used at concentrations ranging preferably from 0.01 to 30% by weight relative to the total weight of the nanoemulsion, and better still from 5 to 20% by weight relative to the total weight of the nanoemulsion. The amount of alcohol(s) and/or of sugar(s) preferably ranges from 5 to 20% by weight relative to the total weight of the nanoemulsion, and the amount of glycol(s) preferably ranges from 5 to 15% by weight relative to the total weight of the nanoemulsion.

The nanoemulsion according to the invention may comprise at least one thickener.

Thickeners can make it possible to increase the viscosity of fluid nanoemulsions (5 cP) by least a factor of 5, for a concentration of polymer equal to 1% by weight. When added to a nanoemulsion, they make it possible to obtain transparent stable compositions constituting milks or creams. The term “milk” or “cream” is intended to mean compositions having a viscosity ranging from 0.5 to 150 poises (i.e. 0.05 Pa·s to 15 Pa·s), measured at 25° C. with a Rheomat 180 with spindle 3, 4 or 5 (depending on the viscosity range), at 200 s⁻¹.

The thickener can be chosen from esters or ethers of polyethylene glycol having from 80 to 350 ethylene oxide units, anionic polymers comprising at least one hydrophobic chain, water-soluble nonionic polymers chosen from ethylene oxide homopolymers and copolymers; polyvinyl alcohols, vinylpyrrolidone homopolymers and copolymers; vinylcaprolactam homopolymers and copolymers; polyvinyl methyl ether homopolymers and copolymers; neutral acrylic homopolymers and copolymers; C₁-C₂ alkylcelluloses and derivatives thereof; C₁-C₃ alkyl-guar or C₁-C₃ hydroxyalkyl-guar.

The thickener may be present in the nanoemulsion according to the invention at a content ranging from 0.005% to 20% by weight, relative to the total weight of the composition, preferably ranging from 0.01 to 10% by weight, and preferentially ranging from 0.1 to 5% by weight.

In particular, the thickeners that are esters or ethers of polyethylene glycol can be chosen from those corresponding to formula (XI) below:
R1-(O—CH2—CH2)n—OR2 (XI)
in which:
R1 denotes a linear or branched, saturated or unsaturated alkyl group or acyl group having from 8 to 30 carbon atoms,
R2 denotes a hydrogen atom, or a linear or branched, saturated or unsaturated alkyl group or acyl group having from 1 to 30 carbon atoms,
n is a number between 80 and 350.

Preferably, R1 is an acyl group having from 12 to 20 carbon atoms.

Preferably, R2 is an acyl group having from 12 to 20 carbon atoms.

Preferably, n is a number between 100 and 300.

Preferably, the weight ratio of the hydrophilic part (—(O—CH₂—CH₂)nO) to the hydrophobic part (R1 and/or R2) is between 8 and 1000.

Use is preferably made of a compound of formula (XI) in which R1 and R2 denote an acyl group having from 12 to 20 carbon atoms and n is between 100 and 300. Mention may, for example, be made of PEG-150 distearate and PEG-250 distearate.

Such compounds are in particular sold under the name Emanon 3299R by the company Kao and under the name Kessco PEG 6000 DS by the company Akzo.

The anionic polymeric thickeners comprising at least one hydrophobic chain that are used as a thickener are preferably water-soluble or water-dispersible, i.e. they are soluble in water at a pH greater than 3.5. They comprise at least one hydrophobic chain, they are noncrosslinked and they preferably have a molecular weight ranging from 10 000 to 2 000 000.

The hydrophobic chain(s) of the anionic polymer used is (are) in particular linear or branched, saturated or unsaturated hydrocarbon-based chains having from 6 to 30 carbon atoms, such as alkyl, arylalkyl, alkylaryl, akylene; divalent cycloaliphatic groups, such as, in particular, methylenedicyclohexyl and isophorone; or divalent aromatic groups such as phenylene.

The thickening anionic polymers can be chosen in particular from acrylic or methacrylic acid copolymers, 2-acrylamido-2-methylpropanesulphonic acid copolymers, and mixtures thereof. The acrylic or methacrylic acid copolymers are preferred. The term “copolymers” is intended to mean both copolymers obtained from two types of monomers and those obtained from more than two types of monomers, such as terpolymers obtained from three types of monomers.

The anionic polymers preferably used are obtained by copolymerization of a monomer (a) chosen from α,β-ethylenically unsaturated carboxylic acids (monomer a′) and 2-acrylamido-2-methylpropanesulphonic acid (monomer a″), with a non-surfactant ethylenically unsaturated monomer (b) that is different from (a) and/or an ethylenically unsaturated monomer (c) derived from the reaction of an α,β-monoethylenically unsaturated acrylic monomer or of a monoethylenically unsaturated isocyanate monomer with a monohydric nonionic amphiphilic component or with a primary or secondary fatty amine.

Thus the anionic polymers used can be obtained by two synthetic pathways:

either by copolymerization of the monomers (a′) and (c) or (a′), (b) and (c), or (a″) and (c), or (a″), (b) and (c),

or by modification (and in particular esterification or amidation) of a copolymer formed from monomers (a′) or from monomers (a′) and (b), or (a″) and (b), with a monohydric nonionic amphiphilic compound or a primary or secondary fatty amine.

As 2-acrylamido-2-methylpropanesulphonic acid copolymers, mention may in particular be made of those described in the article “Micelle formation of random copolymers of sodium 2-(acrylamido)-2-methylpropane-sulfonate and a non-ionic surfactant macromonomer in water as studied by fluorescence and dynamic light scattering—Macromolecules 2000, Vol. 33, No. 10-3694-3704” and in applications EP-A-0 750 899 and EP-A-1,069,172.

The α,β-monoethylenically unsaturated carboxylic acid constituting the monomer (a′) can be chosen from many acids, and in particular from acrylic acid, methacrylic acid, crotonic acid, itaconic acid and maleic acid. It is preferably acrylic acid or methacrylic acid.

The copolymer may comprise a mono-ethylenically unsaturated monomer (b) that does not have any surfactant property. The preferred monomers are those that give water-insoluble polymers when they are homopolymerized. They can be chosen, for example, from C₁-C₄ alkyl acrylates and methacrylates, such as methyl acrylate, ethyl acrylate, butyl acrylate or the corresponding methacrylates. The monomers more particularly preferred are methyl acrylate and ethyl acrylate. The other monomers that can be used are, for example, styrene, vinyltoluene, vinyl acetate, acrylonitrile and vinylidene chloride. Non-reactive monomers are preferred, these monomers being those in which the single ethylene group is the only group that is reactive under the polymerization conditions. However, monomers which comprise groups that are reactive under the action of heat, such as hydroxyethyl acrylate, can optionally be used.

The monomer (c) is obtained by reaction of an α,β-monoethylenically unsaturated acrylic monomer such as (a), or of a monoethylenically unsaturated isocyanate monomer, with a monohydric nonionic amphiphilic compound or a primary or secondary fatty amine.

The monohydric nonionic amphiphilic compounds or the primary or secondary fatty amines used to obtain the nonionic monomer (c) are well known. The monohydric nonionic amphiphilic compounds are generally alkoxylated hydrophobic compounds comprising an alkylene oxide forming the hydrophilic part of the molecule. The hydrophobic compounds generally consist of an aliphatic alcohol or an alkylphenol in which a carbon-based chain containing at least six carbon atoms constitutes the hydrophobic part of the amphiphilic compound.

The preferred monohydric nonionic amphiphilic compounds are compounds having formula (XII) below:
R—(OCH₂CHR′)ₘ—(OCH₂CH₂)ₙ—OH (XII)
in which R is chosen from alkyl or alkylene groups comprising from 6 to 30 carbon atoms, and alkylaryl groups having alkyl radicals comprising from 8 to 30 carbon atoms, R′ is chosen from alkyl groups having from 1 to 4 carbon atoms, n is an average number ranging from approximately 1 to 150 and m is an average number ranging from approximately 0 to 50, on the condition that n is at least as great as m.

Preferably, in the compounds of formula (XII), the group R is chosen from alkyl groups comprising from 12 to 26 carbon atoms and (C₈-C₁₃)alkylphenyl groups; the group R′ is a methyl group; m=0 and n=1 to 25.

The preferred primary and secondary fatty amines consist of one or two alkyl chains comprising from 6 to 30 carbon atoms.

The monomer used to form the nonionic urethane monomer (c) can be chosen from very varied compounds. Any compound comprising a copolymerizable unsaturation such as an acrylic, methacrylic or allyl unsaturation, can be used. The monomer (c) can be obtained in particular from a monoethylenically unsaturated isocyanate such as, in particular, α,α-dimethyl-m-isopropenylbenzyl isocyanate.

The monomer (c) can in particular be chosen from acrylates, methacrylates or itaconates of an oxyethylenated C₆-C₃₀ fatty alcohol (1 to 50 EO), such as steareth-20 methacrylate, oxyethylenated behenyl methacrylate (25 EO), oxyethylenated monocetyl itaconate (20 EO), oxyethylenated monostearyl itaconate (20 EO), or acrylate modified with polyoxyethylenated C₁₂-C₂₄ alcohols (25 EO), and from dimethyl m-iso-propenylbenzylisocyanates of an oxyethylenated C₆-C₃₀ fatty alcohol (1 to 50 EO), for instance oxyethylenated behenyl alcohol dimethyl m-isopropenylbenzylisocyanate.

According to a particular embodiment of the invention, the anionic polymer is chosen from acrylic terpolymers obtained from (a) an α,β-ethylenically unsaturated carboxylic acid, (b) a nonsurfactant ethylenically unsaturated monomer different from (a), and (c) a nonionic urethane monomer which is the product of reaction of a monohydric nonionic amphiphilic compound with a monoethylenically unsaturated isocyanate.

As anionic polymers comprising at least one hydrophobic chain, that can be used in the nanoemulsion of the invention, mention may in particular be made of the terpolymer acrylic acid/ethyl acrylate/alkyl acrylate, such as the product in a 30% aqueous dispersion sold under the name Acusol 823 by the company Rohm & Haas; the acrylate/steareth-20 methacrylate copolymer such as the product sold under the name Aculyn 22 by the company Rohm & Haas; the (meth)acrylic acid/ethyl acrylate/oxyethylenated behenyl methacrylate (25 EO) terpolymer such as the product as an aqueous emulsion sold under the name Aculyn 28 by the company Rohm & Haas; the acrylic acid/oxyethylenated monocetyl itaconate (20 EO) copolymer such as the product as a 30% aqueous dispersion sold under the name Structure 3001 by the company National Starch; the acrylic acid/oxyethylenated monostearyl itaconate (20 EO) copolymer such as the product as a 30% aqueous dispersion sold under the name Structure 2001 by the company National Starch; the copolymer acrylate/acrylate modified with polyoxyethylenated C₁₂-C₂₄ alcohols (25 EO), such as the latex containing 30-32% of copolymer, sold under the name Synthalen W2000 by the company 3V SA; the methacrylic acid/methyl acrylate/ethoxylated behenyl alcohol dimethylmeta-isopropenyl benzylisocyanate terpolymer such as the product as a 24% aqueous dispersion and comprising 40 oxyethylenated groups, described in document EP-A-0 173 109.

The addition of neutralizing agents may prove to be useful for increasing the water-solubility of the polymers. Any known neutralizing agent may then be used, and in particular it can be chosen from inorganic bases such as sodium hydroxide, potassium hydroxide or ammonia, and from organic bases such as mono-, di- and triethanolamine, aminomethylpropanediol-1,3, N-methyl-glucamine, and basic amino acids such as arginine and lysine, and mixtures thereof. The pH of the compositions according to the invention preferably are greater than 4, and more preferably ranges from 5 to 8, and better still from 5 to 7. The amount of neutralizing agent depends on the polymer used and on the other constituents of the formula. It may, for example, range from 0.01 to 5%, and better still from 0.05 to 5%, of the total weight of the composition.

As a thickener, use may also be made of a water-soluble nonionic polymer chosen from ethylene oxide homopolymers and copolymers; polyvinyl alcohols; vinylpyrrolidone homopolymers and copolymers; vinyl-caprolactam homopolymers and copolymers; polyvinyl methyl ether homopolymers and copolymers; neutral acrylic homopolymers and copolymers; C₁-C₂ alkyl-celluloses and derivatives thereof; C₁-C₃ alkyl-guar or C₁-C₃ hydroxyalkyl-guar.

The polymers used according to the present invention are water-soluble, i.e. soluble in water, and nonionic, i.e. neutral.

The water-soluble neutral polymers used according to the invention are in particular chosen from the polymers described below and mixtures thereof.

A) Ethylene oxide homopolymers and copolymers having a molar mass of greater than or equal to 10 000 g/mol, and preferably ranging from 10 000 g/mol to 10 000 000 g/mol. They may be chosen from:

(1) poly(ethylene oxide)s having formula (XIII) below:
R—(CH₂—CH₂—O)ₙ—R′ (XIII)
in which R is chosen from hydroxyl (OH), methoxy (OCH₃) and amine (NH₂) groups, R′ is a methyl (CH₃) group or a hydrogen, and n is a number ranging from 220 to 230 000.
(2) copolymers of ethylene oxide and of one or more oxyalkylenated monomers having formula (XIV) below:
—(CHR—CHR′—O)— (XIV)
in which R and R′ are, independently of one another, a hydrogen or an alkyl group containing from 1 to 7 carbon atoms, at least one of R or R′ being an alkyl group.

Among the ethylene oxide homopolymers and copolymers, mention may in particular be made of the products sold under the names Polyox Coagulant (molar mass of approximately 5×10⁶ g/mol) (CTFA name: PEG-115M) and Polyox WSR N-60K CG (CTFA name: PEG-45M) (molar mass of approximately 2×10⁶ g/mol) by the company Amerchol, and also the product sold under the name Carbowax 20M (CTFA name: PEG-350) (molar mass of approximately 2×10⁷ g/mol) by the company Union Carbide.

B) Polyvinyl alcohols, in particular those having an average molar mass ranging from 10 000 g/mol to 500 000 g/mol. These are compounds represented by formula (XV) below: 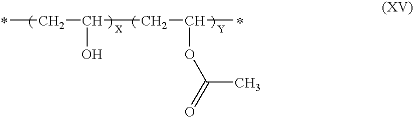
in which x is an average number expressed as a percentage ranging from 70 to 100; y is an average number equal to 100-x.

Mention may, for example, be made of the products sold under the names Airvols 103, 350, 203, 540, 714 and 603 by the company Air Products.

C) Vinylpyrrolidone homopolymers and copolymers, in particular those having an average molar mass ranging from 10 000 g/mol to 1 000 000 g/mol. They can be chosen from:

1) Polyvinylpyrrolidones having formula (XVI) below:

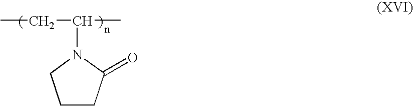

Mention may, for example, be made of the products sold under the names Polyclar V15 (molar mass of approximately 8000 g/mol), V30 (molar mass of approximately 50 000 g/mol), V60 (molar mass of approximately 400 000 g/mol), V90 (molar mass of approximately 1 000 000 g/mol) and V120 (molar mass of approximately 2 500 000 g/mol) by the company ISP.

2) Vinylpyrrolidone copolymers such as:

(a) copolymers of vinylpyrrolidone and of vinyl acetate, in particular the copolymer containing 30% of vinyl acetate, sold under the name PVP-VA 735 by the company ISP;

(b) copolymers of vinylpyrrolidone and of vinylpyrrolidone derivatives with butene grafts, such as the copolymer containing 10% of vinylpyrrolidone with butene grafts sold under the name Ganex (or Antaron) P904 by the company ISP;

(c) copolymers of vinylpyrrolidone and of maleic anhydride (CTFA name: PVM/MA copolymer), such as the products sold under the names Gantrez AN-199 (molar mass of approximately 190 000 g/mol), AN-139 (molar mass of approximately 950 000 g/mol), AN-149 (molar mass of approximately 1 100 000 g/mol), AN-169 (molar mass of approximately 1 700 000 g/mol) and AN-179 (molar mass of approximately 2 000 000 g/mol) by the company ISP;
(d) copolymers of vinylpyrrolidone with polyvinyl alkyl ethers of formula (XVII) below: 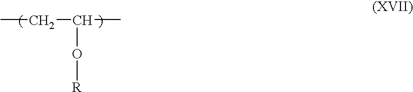
in which R is chosen from alkyl groups containing from 1 to 7 carbon atoms. R is preferably a methyl group;
(e) copolymers of vinylpyrrolidone and of N-vinyllactams such as N-butyrolactam and N-vinylcaprolactam;
(f) copolymers of vinylpyrrolidone with neutral acrylic derivatives of formula (XVIII) below: 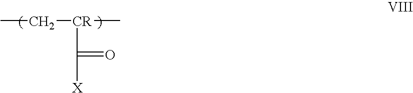
in which R is a hydrogen or a methyl group, and X is chosen from alkyl oxide groups of OR′ type where R′ contains from 1 to 7 carbon atoms; hydroxylated and/or aminated alkyl oxide groups of OR₁(OH)ₙ(NR₂R₃)ₘ type where n and m are numbers ranging from 0 to 10, R₁ is an alkyl group containing from 1 to 7 carbon atoms; R₂ and R₃ are independently hydrogen or an alkyl group such that the sum of the carbon atoms of R₂ and R₃ ranges from 1 to 7; primary, secondary or tertiary amine groups of NR₂R₃ type where R₂ and R₃ have the meaning indicated above.

D) Vinylcaprolactam homopolymers and copolymers, which can be chosen from:
1) polyvinylcaprolactams which have formula (XIX) below: 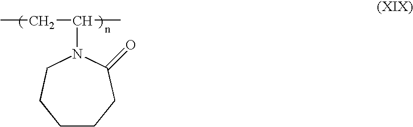
2) vinylcaprolactam copolymers obtained from vinylcaprolactam and one or more of the following monomers:

vinyl acetate;

N-vinyllactam, such as N-butyrolactam, N-vinylcaprolactam and N-vinylpyrrolidone;

maleic anhydride;

vinyl alkyl ethers of formula (XVII) indicated above;

neutral acrylic derivatives of formula (XVIII) indicated above.

As polymers and copolymers of this type, mention may, for example, be made of the product sold under the name Luviskol Plus by the company BASF and the product sold under the name H₂OLD EP-1 by the company ISP.

E) Polyvinyl methyl ether homopolymers and copolymers, which can be chosen from:

1) the polyvinyl methyl ethers of formula (XVII) indicated above;

2) copolymers obtained from vinyl methyl ether and from one or more of the following monomers:

vinyl alkyl ethers of formula (XVII) indicated above;

vinyl acetate;

N-vinyllactam, such as N-butyrolactam, N-vinylcaprolactam and N-vinylpyrrolidone;

maleic anhydride;

neutral acrylic derivatives of formula (XVIII) indicated above.

As polymers and copolymers of this type, mention may, for example, be made of the products sold under the names Gantrez (CTFA name: PVM/MA copolymer), and particularly Gantrez AN-119 (molar mass of approximately 190 000 g/mol), AN-139 (molar mass of approximately 950 000 g/mol), AN-149 (molar mass≈1 000 000 g/mol), AN-169 (molar mass of approximately 1 700 000 g/mol) and AN-179 (molar mass of approximately 2 000 000 g/mol) by the company ISP.

F) Neutral acrylic hompolymers and copolymers, in particular those having a molar mass ranging from 10 000 g/mol to 5 000 000 g/mol. They can be chosen from:
1) neutral water-soluble acrylic polymers having formula (XX) below: 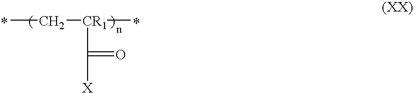
in which R₁ is a hydrogen or a methyl group, and X is chosen from (a) alkylamino groups or (b) hydroxylated and/or aminated alkyl oxide groups.

The polymers with alkylamino groups (a) are compounds of formula (XX) where X=NR₂R₃ such that the corresponding acrylic polymer is water-soluble, R₂ and R₃ being independently a hydrogen or an alkyl group such that the sum of the carbon atoms of R₂ and R₃ ranges from 1 to 7. As polymers of this type, mention may in particular be made of polyacrylamides where R₁, R₂ and R₃ are hydrogen; polymethylacrylamides where R₁ is a methyl group and R₂ and R₃ are a hydrogen; poly-N-methylacrylamides where R₁ and R₂ are a hydrogen and R₃ is a methyl group; poly-N,N′-dimethylacrylamides where R₁ is a hydrogen and R₂ and R₃ are a methyl group; poly-N-ethylacrylamides where R₁ and R₂ are a hydrogen and R₃ is an ethyl group; poly-N-isopropylacrylamides where R₁ and R₂ are a hydrogen and R₃ is an isopropyl group.

As a polymer of this type, mention may be made of the polyacrylamide sold under the name Superfloc N300 LMW by the company Cytec.

The polymers with hydroxylated and/or aminated alkyl oxide groups (b) are compounds of formula (XX) in which X=OR₂(OH)ₙ(NR₃R₄)ₘ where n and m are numbers ranging from 0 to 10, R₂ is an alkyl group containing from 1 to 7 carbon atoms; R₃ and R₄ are independently hydrogen or an alkyl group such that the sum of the carbon atoms of R₃ and R₄ ranges from 1 to 7, these groups being such that the corresponding acrylic derivative is water-soluble.

As a polymer of this type, mention may be made of the poly(glyceryl methacrylate) sold under the name Lubrajel CG by the company Guardian.

2) Copolymers of a water-soluble and neutral acrylic derivative of formula (XX) as described above and of one or more neutral monomer(s) below:

vinyl acetate;

N-vinyllactam, such as N-butyrolactam, N-vinylcaprolactam and N-vinylpyrrolidone;

maleic anhydride;

vinyl alkyl ethers of formula (XVII) indicated above;

a neutral acrylic derivative of formula (XVIII) indicated above.

G) C₁-C₂ alkylcelluloses and neutral derivatives thereof, in particular those having a molar mass ranging from 10 000 g/mol to 5 000 000 g/mol. They can in particular be chosen from hydroxyethylcellulose, such as the product sold under the names Natrosols 250 LR and 250 HHR by the company Aqualon; ethylhydroxyethylcellulose, such as the products sold under the names Elfacos CD 481 and CD 411 by the company Akzo Nobel; methylcellulose and methylhydroxyalkylcelluloses, such as the product sold under the name Methocel A4C by the company Dow Chemical and the products sold under the names Benecel by the company Hercules.

H) C₁-C₃ alkyl-guar or C₁-C₃ hydroxyalkyl-guar, in particular those having a molar mass ranging from 10 000 g/mol to 5 000 000 g/mol. Mention may be made of hydroxypropylguar, such as the product sold under the name Jaguar HP-105 by the company Rhodia.

The method of preparing a nanoemulsion as defined above consists in mixing the aqueous phase containing the urea derivative and the oily phase, with vigorous stirring, at a temperature ranging from 10° C. to 80° C., in carrying out a high-pressure homogenization step at a pressure greater than 5×10⁷ Pa and in optionally adding the polymer used. According to a preferred embodiment of the invention, a high-pressure homogenization step is again subsequently carried out at a pressure of greater than 5×10⁷ Pa. The high-pressure homogenization is preferably carried out at a pressure ranging from 6×10⁷ to 18×10⁷ Pa. The shear preferably ranges from 2×10⁶ s⁻¹ to 5×10⁸ s⁻¹, and better still from 1×10⁸ to 3×10⁸ s⁻¹ (s⁻¹ signifies seconds⁻¹) Such a method makes it possible to prepare nanoemulsions compatible with thermosensitive active compounds, and that may contain oils, and in particular fragrances that contain fatty substances, without denaturing them.

The nanoemulsions defined above can be used in any field where this type of composition is useful. They can especially constitute compositions for topical use, and in particular cosmetic or dermatological compositions according to the type of active agents and the amount of these active agents that they contain. They can also be used as ophthalmic carriers.

They can also constitute, in the pharmaceutical field, the carrier in a pharmaceutical composition that can be administered orally, parenterally or transcutaneously.

Such a composition for topical, pharmaceutical or ophthalmic use contains a physiologically acceptable medium, i.e. a medium that is compatible with the skin, the mucous membranes, the scalp, the eyes and/or the hair.

A subject of the invention is also an ophthalmic carrier, wherein it contains a nanoemulsion as defined above.

A subject of the invention is also a pharmaceutical composition, wherein it contains a nanoemulsion as defined above.

Another subject of the invention consists of a cosmetic or dermatological composition, wherein it consists of a nanoemulsion or comprises a nanoemulsion as defined above.

The compositions of the invention may contain adjuvants, and in particular water-soluble or liposoluble active agents having a cosmetic or dermatological activity. The liposoluble active agents are in the oily globules of the emulsion, whereas the water-soluble active agents are in the aqueous phase of the emulsion. By way of examples of active agents, mention may be made of vitamins and their derivatives, such as vitamin E and its esters, for instance vitamin E acetate, vitamin C and its esters, B vitamins, vitamin A alcohol or retinol and its esters, such as vitamin A palmitate, vitamin A acid or retinoic acid and its derivatives, provitamins such as panthenol and niacinamide, ergocalciferol, antioxidants, essential oils, humectants, sunscreens, moisturizers, proteins, ceramides and pseudoceramides, and DHEA and its derivatives and biological precursors. As adjuvants, mention may also be made of sequestering agents, preserving agents, fillers, UV-screening agents, softeners, dyestuffs (pigments or dyes) and fragrances.

As ophthalmic active agents, mention may, for example, be made of anti-glaucoma agents such as betaxolol; antibiotics such as acyclovir; anti-allergic agents; anti-inflammatories such as ibuprofen and its salts, diclofenac and its salts, indomethacin; antiviral agents.

The amounts of these various adjuvants are not limited and include those conventionally used in the field under consideration, and are, for example, from 0.01% to 20% of the total weight of the composition. These adjuvants and the concentrations thereof should be such that they do not modify the property desired for the composition of the invention.

One subject of the invention is therefore also a cosmetic, dermatological, pharmaceutical or ophthalmological composition comprising a nanoemulsion according to the invention, and in particular a cosmetic composition.

The nanoemulsion of the invention can, for example, be used for caring for, treating and/or making up keratin materials (especially of human beings), in particular the skin (especially the face and/or the scalp) and the lips.

A subject of the invention is also therefore the cosmetic use of the nanoemulsion as defined above, for caring for, treating and/or making up the skin, in particular the face and/or the scalp, and the lips.

The composition according to the invention may be a skincare product, in particular for the face, the neck, the area around the eyes, the body.

The makeup composition comprising the nanoemulsion according to the invention may be a makeup product for the lips (lipstick), a foundation, a blusher, an eyeshadow, an eyeliner, a concealer product, a body makeup product.

Advantageously, the composition is a leave-on composition.

In addition, the nanoemulsion of the invention may also be used for caring for and/or treating the hair. It makes it possible to obtain a deposit of oil on the hair, which gives the latter a greater sheen and makes it more resistant to styling, without, however, making it heavy. It also makes it possible, as a pretreatment, to improve the effects of dyeing or of permanent-waving.

A subject of the invention is therefore also the cosmetic use of the nanoemulsion as defined above, for haircare and/or treatment.

The nanoemulsion according to the invention allows in particular a good moisturization of the skin, the mucous membranes and/or the scalp, and is particularly suitable for treating dry skin.

Another subject of the invention is therefore a cosmetic process for caring for and/or moisturizing and/or making up the skin, the mucous membranes and/or the scalp, wherein a nanoemulsion as defined above is applied to the skin, the mucous membranes and/or the scalp.

The invention also relates to the use of the nanoemulsion according to the invention, for producing a composition for use in the treatment of dry skin.

Finally, the invention also relates to the use of the nanoemulsion according to the invention, for producing an ophthalmological composition.

The following examples will make it possible to understand the invention more clearly, without however being limiting in nature. The amounts indicated are as % by weight unless otherwise mentioned.

The compositions according to the invention can be in the form of an emulsion, in particular of oil-in-water (O/W) or water-in-oil (W/O) or multiple (triple: W/O/W or O/W/O) emulsions. These compositions are prepared according to the usual methods. The composition is preferably an oil-in-water emulsion.

In addition, the compositions used according to the invention may be more or less fluid and may have the appearance of a white or coloured cream, of an ointment, of a milk, of a lotion or of a serum.

The invention is illustrated in greater detail by the nonlimiting examples described hereinafter.

### EXAMPLES

The following procedure is carried out:

in a first phase A, the amphiphilic lipids are homogenized with the oils and the lipophilic active agents and adjuvants at a temperature of approximately 45° C.;

in a second phase B, the hydrophilic active agents and adjuvants are dissolved at a temperature of 20 to 30° C.;

then, phases A and B are mixed using a turbine homogenizer and are then homogenized using a high-pressure homogenizer of the Soavi-Niro type at a pressure of 1200 bar, with 7 passes while maintaining the temperature of the product below 45° C.

### Example 1

A moisturizing care product having the following composition is prepared:

First Phase:

| | |
|---|---|
| PEG-400 isostearate, sold by the company Unichema | 4.5% |
| N-Stearoyl-L-glutamic acid disodium salt sold under the | 0.5% |
| name acylglutamate HS21 by the company Ajinomoto (ionic | |
| amphiphilic lipid) | |
| Jojoba oil | 5% |
| Avocado oil | 5% |
| Cyclomethicone | 9% |
| Nondenatured absolute ethanol | 7.5% |

Second Phase:

| | |
|---|---|
| N-(2-Hydroxyethyl)urea | 7.5% |
| Demineralized water | 61% |

A stable emulsion in which the size of the oil globules is less than 100 nm is obtained.

The composition applied to the skin does not exhibit any feeling of stickiness and provides a good moisturizing effect.

### Example 2

A fragranced water having the following composition is prepared:

First Phase:

| | |
|---|---|
| Mixture of sucrose palmitate/stearate sold under the name | 4.5% |
| Crodesta F70 by the company Croda | |
| N-Stearoyl-L-glutamic acid disodium salt sold under the | 0.5% |
| name acylglutamate HS21 by the company Ajinomoto (ionic | |
| amphiphilic lipid) | |
| Soybean oil | 6% |
| Volatile silicone oil | 2% |
| Fragrance | 3% |
| Vitamin E acetate | 0.5% |
| Nondenatured absolute ethanol | 10% |

Second Phase:

| | |
|---|---|
| N-(2-hydroxyethyl)urea | 5% |
| Demineralized water | 68.5% |

A stable emulsion in which the size of the oil globules is less than 100 nm is obtained.

The composition applied to the skin does not exhibit any feeling of stickiness and provides a good moisturizing effect.

### Example 3

A leave-on makeup-removing milk having the following composition is prepared:

First Phase:

| | |
|---|---|
| Oxyethylenated sorbitan tristearate (20 EO) sold under | 4.5% |
| the name Tween 65 by the company Unichema | |
| N-Stearoyl-L-glutamic acid disodium salt sold under | 0.5% |
| the name acylglutamate HS21 by the company Ajinomoto | |
| (ionic amphiphilic lipid) | |
| Isocetyl stearate | 5% |
| Isopropyl myristate | 10% |
| Cyclomethicone | 5% |
| Nondenatured absolute ethanol | 7.5% |

Second Phase:

| | |
|---|---|
| N-(2-Hydroxyethyl)urea | 7.5% |
| Demineralized water | 61% |

A stable emulsion in which the size of the oil globules is less than 100 nm is obtained.

The composition is applied readily to the skin, exhibits no feeling of stickiness and provides a good moisturizing effect.

The above written description of the invention provides a manner and process of making and using it such that any person skilled in this art is enabled to make and use the same, this enablement being provided in particular for the subject matter of the appended claims, which make up a part of the original description and including an oil-in-water nanoemulsion comprising an oily phase dispersed in an aqueous phase, the oil globules of which have a number-average size of less than 100 nm, wherein it comprises:

(i) at least one amphiphilic lipid comprising at least one nonionic amphiphilic lipid, and optionally an ionic amphiphilic lipid, the oily phase and the amphiphilic lipid being present at a content such that the oily phase/amphiphilic lipid weight ratio ranges from 3 to 10,

and (ii) at least one compound of formula (I) below: 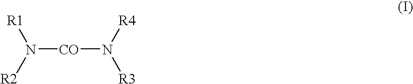
in which:
R₁, R₂, R₃ and R₄ each represent, independently of one another, a hydrogen atom, a C₁-C₄ alkyl group or a C₂-C₆ hydroxyalkyl group that may contain from 1 to 5 hydroxyl groups, where at least one of the radicals R₁ to R₄ represents a hydroxyalkyl group, and also the salts, solvates and isomers thereof.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

As used herein, where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An oil-in-water nanoemulsion comprising an oily phase dispersed in an aqueous phase, the oil globules of which have a number-average size of less than 100 nm, wherein it comprises:
(i) at least one amphiphilic lipid comprising at least one nonionic amphiphilic lipid, optionally at least one ionic amphiphilic lipid, the oily phase and the amphiphilic lipid being present such that the oily phase/amphiphilic lipid weight ratio is 3-10, and
(ii) at least one compound of formula (I) below, including salts, solvates and isomers thereof: 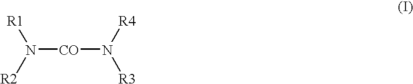
in which:
R₁, R₂, R₃ and R₄ each represent, independently of one another, a hydrogen atom, a C₁-C₄ alkyl group or a C₂-C₆ hydroxyalkyl group that contains from 1 to 5 hydroxyl groups, where at least one of the radicals R₁ to R₄ represents a hydroxyalkyl group.

2. The nanoemulsion according to claim 1, wherein, for the compounds of formula (I), R₁ denotes a C₂-C₆ hydroxyalkyl group, and R₂, R₃ and R₄ denote, independently of one another, a hydrogen atom or a C₁-C₄ alkyl group.

3. The nanoemulsion according to claim 1, wherein, for the compounds of formula (I), R₁ denotes a C₂-C₆ hydroxyalkyl group containing from 1 to 5 hydroxyl groups, and R₂, R₃ and R₄ denote a hydrogen atom.

4. The nanoemulsion according to claim 3, wherein R₁ denotes a C₂-C₆ hydroxyalkyl group containing 1 hydroxyl group.

5. The nanoemulsion according to claim 1, wherein, for the compounds of formula (I), R₁ denotes a C₂-C₄ hydroxyalkyl group containing 1 hydroxyl group, and R₂, R₃ and R₄ denote a hydrogen atom.

6. The nanoemulsion according to claim 1, comprising at least one compound of formula (I) chosen from N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-(trishydroxymethylmethyl)urea; N-ethyl-N′-(2-hydroxyethyl)urea; N,N-bis(2-hydroxyethyl)urea; N,N′-bis(2-hydroxyethyl)urea; N,N-bis(2-hydroxypropyl)-urea; N,N′-bis(2-hydroxypropyl)urea; N,N-bis(2-hydroxyethyl)-N′-propylurea; N,N-bis(2-hydroxypropyl)-N′-(2-hydroxyethyl) urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′,N′-tetrakis(2-hydroxyethyl)urea; N′,N′-bis(2-hydroxyethyl)-N′,N′-bis(2-hydroxypropyl)urea; and mixtures thereof.

7. The nanoemulsion according to claim 1, comprising N-(2-hydroxyethyl)urea.

8. The nanoemulsion according to claim 1, wherein the salts of the compounds of formula (I) are chosen from salts of sulphuric acid, hydrochloric acid, hydrobromic acid, hydriodic acid, phosphoric acid, boric acid, propionic acid, acetic acid, terephthalic acid, citric acid and tartaric acid.

9. The nanoemulsion according to claim 1, wherein the compound of formula (I) is present in an amount of 1% to 50% by weight, relative to the total weight of the composition.

10. The nanoemulsion according to claim 9, wherein the oily phase/amphiphilic lipid weight ratio is 2 to 6.

11. The nanoemulsion according to claim 1, wherein the oil globules have a number-average size of 20 to 80 nm.

12. The nanoemulsion according to claim 1, comprising at least one nonionic amphiphilic lipid chosen from:
1/silicone surfactants,
2/amphiphilic lipids that are liquid at a temperature of less than or equal to 45° C., chosen from esters of at least one polyol and of at least one fatty acid comprising at least one saturated or unsaturated, linear or branched C₈-C₂₂ alkyl chain,
3/fatty acid esters of a sugar and fatty alcohol ethers of a sugar,
4/surfactants that are solid at a temperature equal to 45° C., chosen from glycerol fatty esters, sorbitan fatty esters and oxyethylenated sorbitan fatty esters, ethoxylated fatty ethers and ethoxylated fatty esters,
5/block copolymers of ethylene oxide and of propylene oxide,
and mixtures thereof.

13. The nanoemulsion according to claim 1, wherein the nonionic amphiphilic lipid is present in an amount of 0.2% to 12% by weight, relative to the total weight of the composition.

14. The nanoemulsion according to claim 1, wherein it comprises at least one ionic amphiphilic lipid.

15. The nanoemulsion according to claim 14, wherein comprising at least one ionic amphiphilic lipid chosen from:
alkali metal salts of dicetyl phosphate and of dimyristyl phosphate;
alkali metal salts of cholesterol sulphate;
alkali metal salts of cholesterol phosphate;
lipoamino acids and their salts;
sodium salts of phosphatidic acid;
phospholipids;
alkylsulphonic derivatives;
quaternary ammonium salts;
fatty amines and their salts.

16. The nanoemulsion according to claim 14, wherein the ionic amphiphilic lipid is present in an amount of 0.01% to 6% by weight relative to the total weight of the composition.

17. The nanoemulsion according to claim 1, wherein the total content of nonionic and ionic amphiphilic lipids is 0.25% to 15% by weight, relative to the total weight of the composition.

18. The nanoemulsion according to claim 1, wherein the amount of oily phase is 2% to 40% by weight relative to the total weight of the composition.

19. The nanoemulsion according to claim 1, comprising at least one oil having a molecular weight of 400 to 10 000.

20. The nanoemulsion according to claim 19, wherein the oily phase comprises a proportion of oil(s) having a molecular weight of 400 to 10 000 representing at least 40% by weight of the oily phase.
